(19) United States
(12) Patent Application Publication
(10) Pub. No.: US 2022/0401446 A1
(43) Pub. Date: Dec. 22, 2022

(54) CONTROLLED RELEASE TOFACITINIB COMPOSITIONS

(71) Applicant: Synthon B.V., Nijmegen (NL)

(72) Inventors: Lisardo ALVAREZ FERNANDEZ, Sant Boi de Llobregat (ES); Luis NOGUEIRAS NIETO, Sant Boi de Llobregat (ES); Rohit KUMAR, Sant Boi de Llobregat (ES)

(21) Appl. No.: 17/638,448

(22) PCT Filed: Aug. 27, 2020
(86) PCT No.: PCT/EP2020/074032

§ 371 Date: Feb. 25, 2022

(30) Foreign Application Priority Data
Aug. 29, 2019 (EP) 19194282.0

(51) Int. Cl. A61K 31/519; A61K 9/28; A61K 9/50; A61K 9/20

(52) U.S. Cl. CPC A61K 31/519; A61K 9/282; A61K 9/5015; A61K 9/2054

(57) ABSTRACT

The present invention relates to a monolithic tablet composition for oral administration of tofacitinib, or a pharmaceutically acceptable salt thereof.

### BACKGROUND OF THE PRESENT INVENTION

Tofacitinib or (3R,4R)-4-methyl-3-(methyl-7H-pyrrolo [2,3-d]pyrimidin-4-ylamino)-ß-oxo-1-piperidinepropanenitrile, citrate salt (1:1), of the formula:

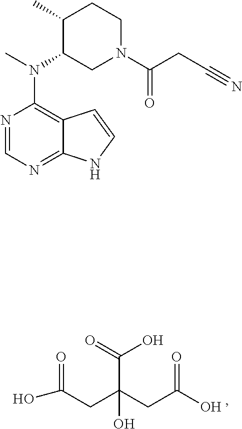 [SMILES: C[C@@H]1CCN(C(=O)CC#N)C[C@@H]1N(C)c1ncnc2[nH]ccc12 ; O=C(O)CC(O)(CC(=O)O)C(=O)O]

is a reversible inhibitor of the Janus kinase family of kinases (JAK1, JAK2, JAK3 and Tyrosine Kinase 2 (TyK2)). Tofacitinib has been disclosed in WO2001042246.

Tofacitinib is indicated for the treatment of adult patients with moderately to severely active rheumatoid arthritis who have had an inadequate response or intolerance to methotrexate. It is marketed as an extended release tablet under the brand name XELJANZ XR® (Pfizer Products Inc.). The tablets are based on osmotic pump technology, wherein the osmotic pressure is used to deliver the tofacitinib at controlled rate. The tablet insert for XELJANZ XR® tablet, describes the tablet as “a pink, oval, extended release film-coated tablet with a drilled hole at one end of the tablet band”.

XELJANZ XR® tablet is a controlled-release formulation, which provides more favourable pharmacokinetic profiles (e.g. reducing the peak variation of drug concentration levels), so reducing the side effects and achieving better patient compliance.

XELJANZ XR® drug release profile is very complicated combining different order kinetics. XELJANZ XR® formulation is described in WO2014147526; the formulation is an osmotic pump consisting of a coating made of an insoluble polymer, cellulose acetate, and a core containing tofacitinib citrate, sorbitol, hydroxyethyl cellulose, co-povidone and magnesium stearate. This coating is such that tofacitinib is substantially entirely delivered through the delivery hole, in contrast to delivery via permeation through the coating. The solute concentration gradient, which provides the osmotic force driving the delivery of the drug through the drilled hole, can be maintained constant when solute saturation is present in the tablet core. As the tablet content comes out, solute concentration declines and as well the gradient and the osmotic force driving the drug release.

The typical orifice size in osmotic pumps ranges from about 600 μm to 1 mm. A nominal 600 μm hole usually has a ±100 μm tolerance on diameter, and an allowable ellipticity of 1.0 to 1.5. Although holes of these characteristics and tolerances can be obtained by mechanical means, there is no mechanical method able to work at high manufacturing rates consistent with pharmaceutical manufacturing processes.

In contrast, laser tablet drilling can lead to throughput rates of up to 100,000 tablets/hour having the necessary dimensional tolerances and cosmetic appearance. As a result, laser drilling has become the technology of choice for this type of orifice production.

This technology also requires accepted-rejected system in order to check if the drilled hole on the surface of the tablet meets the specifications. The reject mode is activated as soon as a failed tablet is sensed by the vision system, which causes one or two tablets ahead of the rejected unit to be expelled as well. The reject state only switches off when the system verifies that five tablets in a row meet pass criterion. An additional presence sensor downstream from the blow off verifies that no tablets are passing through the system when the reject condition is set to “on”.

Therefore, the required technology for the manufacturing of the osmotic pump delivery systems is significantly expensive, which is a disadvantage and an economic barrier for many companies.

WO 2012/100949 provides an oral dosage form for modified release comprising tofacitinib and a non-erodible material. In this patent application a monolithic tablet containing a non-erodible material and other components such as pore formers is claimed. The main disadvantage of this type of delivery systems is the difficulties of the water to penetrate through the material, leading to slow hydration rates. This may lead as result the incomplete dissolution of the drug substance if the centre of the tablet core remains unwetted.

WO 2014/174073A1 discloses a sustained release formulation for oral administration comprising tofacitinib, a hydrophilic polymer and an alkalizing agent. The alkalizing agent is proposed for reducing API solubility in acidic pHs obtaining a non-pH dependent release formulation. Alkalizing the tablet core aims to reduce the release of the active ingredient at low pHs where it is more soluble; however, the decrease of the active ingredient solubility by alkalizing the tablet core can limit the drug release at high pHs (for instances at the small intestine) impacting on the bioavailability of the drug substance.

There is still need of finding an additional oral formulation of tofacitinib which overcome the problems of the prior art and is bioequivalent to the commercial tofacitinib tablet XELJANZ XR®.

### DETAILED DESCRIPTION OF THE PRESENT INVENTION

The present invention relates to a monolithic tablet that is able to provide a similar dissolution release rate of tofacitinib than the commercial tables having an osmotic pump.

As used herein the term “monolithic tablet” refers to a tablet comprising a swellable hydrophilic matrix that delivers the drug in a controlled manner over a long period of time.

A first aspect of the invention relates to a controlled release pharmaceutical tablet comprising:
a) A core comprising tofacitinib or a pharmaceutically acceptable salt thereof and a pH independent gelling control release polymer;
b) A coating in an amount of 1.5% to 10.0% w/w in relation to the total tablet weight comprising a water-insoluble polymer and a pore former in a weight ratio of from 90:10 w/w to 60:40 w/w respectively.

The dissolution profile provided by the osmotic pump of tofacitinib marketed tablet initially it exhibits a short lag time where no drug release takes place. This short lag time corresponds with the diffusion of water through the semi-permeable membrane and the hydration of the tablet core. Afterwards, zero-order kinetic release occurs due to the sustained solute concentration gradient between the tablet core and the dissolution medium. The solute concentration gradient, which provides the osmotic force driving the delivery of the drug through the drilled hole, can be maintained constant whereas solute saturation takes place in the tablet core. As the tablet content come out, the solute concentration declines and so the gradient and the osmotic force driving drug release. Ultimately, as a consequence of the decrease of the solute concentration in the tablet core, the dissolution profile shows first-order kinetic release after 3 hours.

Hydrophilic matrix technology has been widely used for oral controlled delivery of various drugs. As well the combination of barrier membrane and hydrophilic matrix system has been utilized as a strategy to modulate drug release from hydrophilic matrices and to reduce the overall variability in release. However, it is difficult particularly for very soluble compounds to apply this technology and achieve zero order release. We have surprisingly found that in the case of tofacinib, by applying a coating with a specific coating amount (measured by weight gain of the total tablet) that comprises a water-insoluble polymer and a pore former in a particular ratio results in a zero-order release.

The monolithic tablet of the current invention provides similar drug dissolution release to an osmotic pump system by creating multiple pores that allows the constant diffusion of tofacitinib through the coating. Moreover, the technology required for the manufacturing of a monolithic tablet is cheaper and as efficient as the one employed for obtaining osmotic pump systems.

The monolithic tablet of the present invention comprises a core and a coating. The core comprising Tofacitinib and a pH independent gelling control release polymer while the coating comprises a water-insoluble polymer and a pore former.

It was surprisingly found that the weight ratio between the water-insoluble polymer and the pore former of the coating and the weight gain of the tablet strongly influences the dissolution profile of the monolithic tablet of the current invention. To fine-tune the dissolution, the viscosity of the polymers can be varied.

The core of the controlled release pharmaceutical tablet of the invention comprises the whole dose of tofacitinib. The word tofacitinib is used herein to refer to tofacitinib free base as well as its pharmaceutically acceptable salts. A preferred salt to be used is the citrate salt.

Tofacitinib free base as well as its pharmaceutically acceptable salts, preferably tofacitinib citrate, is preferably used in an amount of 3% to 15%, more preferably 4% to 12%, most preferably 7% to 10% by weight based on the total inner tablet weight.

In the present invention tofacitinib is released from the formulation, in a controlled fashion so that at least 60% of tofacitinib is released after 4 hours and at least 80% of tofacitinib is released after 6 hours.

In the present invention the core of the tablet contains at least one pH independent gelling control release polymer. The term pH independent gelling control release polymer means a control release polymer that forms a gel when in contact with water independently of the pH of the water. Such polymers are known in the art and include polyethylene oxide (for example (MW:900.000 g/mol; Polyox® 1105 WSR)), hydroxypropyl methylcellulose (for example Methocel® K100 Premium low viscosity (LV) grade), hydroxypropyl cellulose, polyvinyl alcohol (for example Parteck® SRP 80), guar gum, carrageenan and combinations thereof. A preferred pH independent gelling control release polymers are soluble polymers such a polyethylene oxide, hydroxypropyl methylcellulose, hydroxypropyl cellulose, polyvinyl alcohol and combinations thereof. More preferably a pH independent gelling control release polymers are polyethylene oxide and hydroxypropyl methyl cellulose, even more preferably a pH independent gelling control release polymers is hydroxypropyl methyl cellulose. The amount of the pH independent gelling control release polymer in the tablet core is preferably in an amount from 10% to 50%, more preferably from 10% to 40%, even more preferably 10% to 30% by weight based on the total tablet core weight.

The pH independent gelling control release polymer of the present invention has preferably a viscosity of 10 cP or more, more preferably 20 cP or more, even more preferably between 20 and 500 cP, most preferred 24 to 300 cP in a solution containing 2% of the polymer in distilled water at 22.5±0.5° C., measured using a rotational viscometer as Fungilab viscometer.

The tablet core may contain additional excipients such as diluents, binders, lubricants, glidant or buffering agents.

Diluents are excipients that are used to increase the bulk volume of a tablet. By combining a diluent with the active pharmaceutical ingredient, the final product is given adequate weight and size to assist in production and handling.

The tablet core of the present invention contains preferably at least one diluent. Diluents are preferably used in an amount of from 40% to 90% more preferably 50 to 80% most preferably 60% to 80% by weight based on the total weight of the tablet core. Suitable examples of diluents to be used in accordance with the present invention include lactose, starch, pregelatinized starch, microcrystalline cellulose (MCC), phosphates, and combinations thereof.

In a preferred embodiment of the present invention, the diluents to be used are lactose, microcrystalline cellulose or mixtures thereof.

Binders hold the excipients that are present in a tablet together. Binders ensure that tablets and granules can be formed having the desired or required mechanical strength.

Binders which are suitable for use in accordance with the present invention include povidone, hydroxypropyl methylcellulose, hydroxy propylcellulose, and sodium carboxyl methylcellulose. Binders are preferably used in an amount of from 1% to 10% by weight based on the total weight of the tablet core. A preferred binder is hydroxypropyl cellulose, povidone or co-povidone.

The tablet core may also contain a lubricant and/or a glidant. Lubricants are generally used in order to reduce sliding friction. In particular, to decrease friction at the interface between a tablet's surface and the die wall during ejection, and reduce wear on punches and dies. Suitable lubricants to be used in accordance with the present invention include magnesium stearate, calcium stearate, stearic acid, glyceryl behenate, hydrogenated vegetable oil, and sodium stearyl fumarate. Lubricants are preferably used in a total amount of from 0.05% to 5%, more preferably 0.5% to 2%, most preferably 0.8% to 1.5% by weight based on the total weight of the tablet core. A preferred lubricant is magnesium stereate.

Glidants enhance product flow by reducing interparticulate friction. A suitable example is colloidal silicon dioxide. Glidants are preferably used in a total amount of from 0.05% to 5%, more preferably 0.2% to 2%, most preferably 0.2% to 1.0% by weight based on the total weight of the tablet core.

The tablet core may also contain one or more buffering agents. Buffering agents are generally used in order to maintain the pH constant. They may be acidic or basic agents. Suitable acidic buffering agents are tartaric acid, malic acid, maleic acid and citric acid. Suitable basic buffering agents are sodium carbonate, sodium acetate and potassium citrate.

In the present invention the tablet core is coated with a coating which comprises at least one water-insoluble polymer and at least one pore former.

The term “water-insoluble polymer” as used herein refers to a polymer having a solubility in water lower than 0.05 g/100 ml water, measured at 20° C. at 1 atm pressure. The water-insoluble polymer functions as controlled release of the drug that is in the core. The term “pore former”, as used herein, refers to a material added to the coating solution that has low or no volatility relative to the solvent such that it remains as part of the coating following the coating process but that is sufficiently water swellable or water soluble such that, in the aqueous use environment it provides a water-filled or water-swollen channel or “pore” to allow water penetration and the release of the drug that is in the core.

Suitable water-insoluble polymers for the coating are ethylcellulose, cellulose acetate, methacrylic ester copolymers, polyvinyl acetates. In a preferred embodiment of the present invention, the water-insoluble polymer is ethylcellulose.

Suitable pore former for the tablet of the current invention are hydroxypropyl cellulose, hydroxypropyl methylcellulose, methylcellulose, polyethylene glycol, polyethylene oxide, polyvinyl alcohol, povidone, maltodextrin, saccharides such as glucose, isomalt, sucrose, polyols such as maltitol, xylitol. In a preferred embodiment of the present invention, the pore former is hydroxypropyl methylcellulose.

The preferred weight ratio of the water-insoluble polymer and the pore former in the coating is from 90:10 to 40:60. More preferably the weight ratio of the water-insoluble polymer and the pore former is from 90:10 to 70:30. Most preferred the weight ratio of the water-insoluble polymer and the pore former is from 90:10 to 75:25.

Further the tablet of the invention has a coating in an amount of 1.5% to 10.0% w/w in relation to the total tablet weight and it shows a dissolution profile similar and it is bioequivalent to the commercial tofacitinib tablet XELJANZ XR®.

Preferably, the total tablet weight increase percentage ranges from 5% to 10%, more preferably 2% to 8% w/w, most preferably from 4% to 6% w/w, even most preferably from 3% to 5% w/w.

The coating may be prepared by mixing the pore former with water obtaining a homogeneous solution. The obtained solution is mixed with the water insoluble polymer; optionally other excipients like plasticizer, colourants etc. are added obtaining a homogeneous suspension.

Typically, a suitable commercial coating base is Surelease®. Surelease® is an aqueous ethylcellulose dispersions containing between 17% and 20% of ethyl cellulose, plasticizers and stabilizers which is ready to be mixed with the pore former solution obtaining a homogeneous suspension. The obtained suspension is sprayed over the tablets.

In a preferred embodiment the tablet comprises:
1. A core comprising:
   a. Tofacitinib or a pharmaceutically acceptable salt, preferably tofacitinib citrate in an amount of from 3% to 15% w/w based on the total weight of the core weight;
   b. Hydroxypropyl methylcellulose in an amount of from 10% to 50% w/w based on the total weight of the core weight;
   c. One or more diluents in an amount of from 40% to 90% w/w based on the total weight of the core weight;
   d. Glidant in an amount of from 0.2% to 1.0% w/w based on the total weight of the core weight;
   e. Lubricant of from 0.05% to 5% w/w based on the total weight of the core weight; and
2. A coating comprising ethylcellulose and hydroxypropyl methylcellulose in a weight ratio of from 90:10 w/w to 60:40 w/w based on the total tablet weight.

In a more preferred embodiment the tablet comprises:
1. A core comprising:
   a. Tofacitinib or a pharmaceutically acceptable salt, preferably tofacitinib citrate in an amount of from 3% to 15% w/w based on the total weight of the core weight;
   b. Hydroxypropyl methylcellulose in an amount of from 10% to 50% w/w based on the total weight of the core weight;
   c. Microcrystalline cellulose and lactose monohydrate in an amount of from 40% to 90% w/w based on the total weight of the core weight;
   d. Colloidal silicon dioxide in an amount of from 0.2% to 1.0% w/w based on the total weight of the core weight;
   e. Magnesium stearate of from 0.05% to 5% w/w based on the total weight of the core weight; and
2. A coating comprising ethylcellulose and hydroxypropyl methylcellulose in a weight ratio of from 90:10 w/w to 60:40 w/w.

In a most preferred embodiment the tablet of the invention comprises:
1. A core comprising:
   a. Tofacitinib or a pharmaceutically acceptable salt, preferably tofacitinib citrate in an amount of from 5% to 10% w/w based on the total weight of the core weight;
   b. Hydroxypropyl methylcellulose in an amount of from 10% to 20% w/w based on the total weight of the core weight;
   c. One or more diluents in an amount of from 60% to 80% w/w based on the total weight of the core weight; preferably lactose
   d. Colloidal silicon dioxide in an amount of from 0.5% to 1.0% w/w based on the total weight of the core weight;
   e. Magnesium stearate of from 1% to 3% w/w based on the total weight of the core weight; and
2. A coating comprising ethylcellulose and hydroxypropyl methylcellulose in a weight ratio of from 90:10 w/w to 60:40 w/w.

The tablet of the invention can be made using conventional methods and equipment well-known in the art; direct compression, wet granulation or dry granulation. In a preferred embodiment the tablet of the invention is prepared by direct compression.

The tablet composition in accordance with the present invention is bioequivalent in vitro and in vivo to the commercially available tofacitinib citrate tablets.

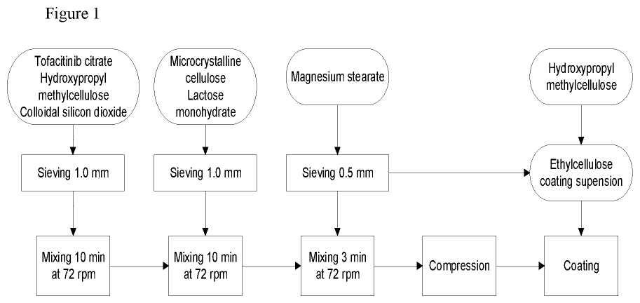

FIG. 1 shows the manufacturing scheme of the formulation.

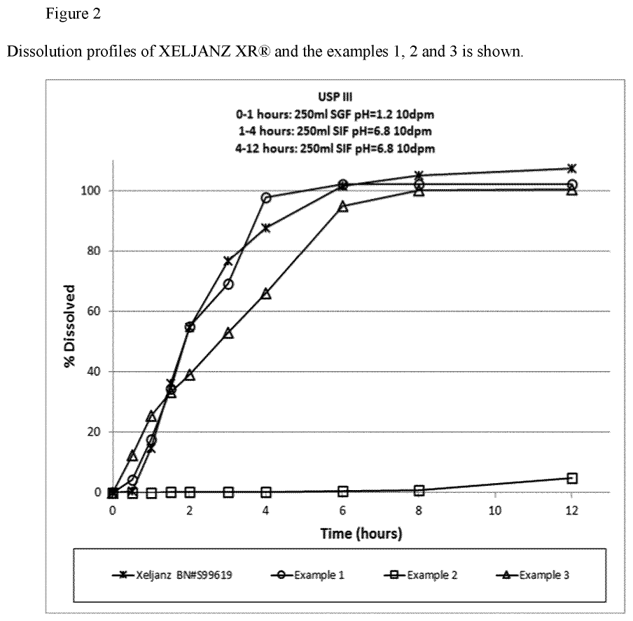

FIG. 2 shows the in vitro dissolution profile of tablet compositions in accordance with the present invention as compared to commercially available tablets.

The present invention is illustrated by the following Examples. In table 1 the pharmaceutical composition of examples 1, 2 and 3 are shown.

### Example 1: Controlled Release Formulation Containing 15% w/w of Methocel K100 LV in the Tablet Core, Coating Weight Increase of 5.0% Containing a Ratio of Ethyl Cellulose:Hydroxypropyl Methylcellulose 81.5:18.5

35.5 grams of tofacitinib citrate, 60.0 grams of Methocel K100 LV (Hydroxypropyl methylcellulose) and 2.0 grams of Aerosil VV Pharma (Colloidal silicon dioxide) are weighed and deagglomerated through a sieve of 1.0 mm mesh size. The components are mixed in a Turbula blender at 72 rpm for 10 minutes obtaining a homogenous blend (1). 149.2 grams of microcrystalline cellulose and 149.2 grams of lactose monohydrate are weighed, deagglomerated through a sieve of 1.0 mm mesh size and then added together with the previous blend (1); the components are mixed in a Turbula blender at 72 rpm for 10 minutes, obtaining a homogenous blend (2). 4 grams of Magnesium stearate are weighed and deagglomerated using a sieve of 0.5 mm mesh size and added to the previous blend (2); the components are mixed in a Turbula blender at 72 rpm for 3 minutes, resulting in a homogenous blend (3). This blend (3) is then compressed in a rotatory tabletting machine, obtaining tablets with appropriate hardness (≈150 N).

The coating suspension is prepared in excess (120%) for the coating of the tablets.

3.4 grams of Methocel E5 LV are weighed and added into 114.4 grams of distilled water, mixed with a magnetic stirrer until its total dissolution, obtaining a homogenous solution (4).

Then the previous solution (4) is poured into 82.2 grams of Surelease® and stirred during 45 minutes before starting the coating process, resulting in a homogenous suspension (5). Then the suspension (5) is sprayed over the tablets previously heated in the coating pan, until the tablets achieved a 5.0% w/w weight increase.

### Comparative Example 2: Controlled Release Formulation Containing 20% w/w of Methocel K100 LV in the Tablet Core, Coating Weight Increase of 5.0% Containing a Ratio of Ethyl Cellulose:Hydroxypropyl Methylcellulose 95.0:5.0

35.5 grams of tofacitinib citrate, 80.0 grams of Methocel K100 LV (Hydroxypropyl methylcellulose) and 2.0 grams of Aerosil VV Pharma (Colloidal silicon dioxide) are weighed and deagglomerated through a sieve of 1.0 mm mesh size. The components are mixed in a Turbula blender at 72 rpm for 10 minutes obtaining a homogenous blend (1). 139.2 grams of microcrystalline cellulose and 139.2 grams of lactose monohydrate are weighed, deagglomerated through a sieve of 1.0 mm mesh size and then added together with the previous blend (1); the components are mixed in a Turbula blender at 72 rpm for 10 minutes, obtaining a homogenous blend (2). 4 grams of Magnesium stearate are weighed and deagglomerated using a sieve of 0.5 mm mesh size and added to the previous blend (2); the components are mixed in a Turbula blender at 72 rpm for 3 minutes, resulting in a homogenous blend (3). This blend (3) is then compressed in a rotatory tabletting machine, obtaining tablets with appropriate hardness (≈150 N).

The coating suspension is prepared in excess (120%) for the coating of the tablets.

0.9 grams of Methocel E5 LV are weighed and added into 106.7 grams of distilled water, mixed with a magnetic stirrer until its total dissolution, obtaining a homogenous solution (4).

Then the previous solution (4) is poured into 92.4 grams of Surelease® and stirred during 45 minutes before starting the coating process, resulting in a homogenous suspension (5). Then the suspension (5) is sprayed over the tablets previously heated in the coating pan, until the tablets achieved a 5.0% w/w weight increase.

### Comparative Example 3: Controlled Release Formulation Containing 20% w/w of Methocel K100 LV in the Tablet Core, Coating Weight Increase of 5.0% Containing a Ratio of Ethyl Cellulose:Hydroxypropyl Methylcellulose 55.0:45.0

35.5 grams of tofacitinib citrate, 80.0 grams of Methocel K100 LV (Hydroxypropyl methylcellulose) and 2.0 grams of Aerosil VV Pharma (Colloidal silicon dioxide) are weighed and deagglomerated through a sieve of 1.0 mm mesh size. The components are mixed in a Turbula blender at 72 rpm for 10 minutes obtaining a homogenous blend (1). 139.2 grams of microcrystalline cellulose and 139.2 grams of lactose monohydrate are weighed, deagglomerated through a sieve of 1.0 mm mesh size and then added together with the previous blend (1); the components are mixed in a Turbula blender at 72 rpm for 10 minutes, obtaining a homogenous blend (2). 4 grams of Magnesium stearate are weighed and deagglomerated using a sieve of 0.5 mm mesh size and added to the previous blend (2); the components are mixed in a Turbula blender at 72 rpm for 3 minutes, resulting in a homogenous blend (3). This blend (3) is then compressed in a rotatory tabletting machine, obtaining tablets with appropriate hardness (≈150 N).

The coating suspension is prepared in excess (120%) for the coating of the tablets.

9.1 grams of Methocel E5 LV are weighed and added into 131.2 grams of distilled water, mixed with a magnetic stirrer until its total dissolution, obtaining a homogenous solution (4).

Then the previous solution (4) is poured into 59.8 grams of Surelease® and stirred during 45 minutes before starting the coating process, resulting in a homogenous suspension (5). Then the suspension (5) is sprayed over the tablets previously heated in the coating pan, until the tablets achieved a 5.0% w/w weight increase.

TABLE 1

| | | |
|---|---|---|
| Example 1 | Example 2 | Example 3 |

Tablet core

| Components | mg/tablet | mg/tablet | mg/tablet |
|---|---|---|---|
| TOFACITINIB citrate | 17.76 | 17.76 | 17.76 |
| Hydroxypropyl | 30 | 40 | 40 |
| methylcellulose | | | |
| Colloidal silicon dioxide | 1 | 1 | 1 |
| Microcrystalline cellulose | 74.62 | 69.62 | 69.62 |
| Lactose monohydrate | 74.62 | 69.62 | 69.62 |
| Magnesium stearate | 2 | 2 | 2 |
| Total tablet core weight (mg) | 200 | 200 | 200 |

Coated tablet

| | | Ratio | | Ratio | | Ratio |
|---|---|---|---|---|---|---|
| Components | mg/tablet | EC:HPMC | mg/tablet | EC:HPMC | mg/tablet | EC:HPMC |
| Ethyl cellulose (EC) | 6.31 | 81.5 | 7.09 | 95.0 | 4.59 | 55.0 |
| Hydroxypropyl | 1.43 | 18.5 | 0.37 | 5.0 | 3.76 | 45.0 |
| methylcellulose (HPMC) | | | | | | |

| | mg/tablet | mg/tablet | mg/tablet |
|---|---|---|---|
| Additional solids from | 2.26 | 2.54 | 1.65 |
| Surelease dispersion | | | |
| Total coated tablet weight | 210 | 210 | 210 |
| (mg) | | | |

The above formulations were made according to the process depicted in FIG. 1.

The ratio between ethyl cellulose (EC) and hydroxypropyl methylcellulose (HPMC) is of 81.5 to 18.5 in example 1, of 95.0 to 5.0 in example 2 and of 55.0 to 45.0 in example 3.

The EC is used as Surelease suspension (Surelease®), this suspension comprises EC and other excipients (additional solids).

The formulations of table 1 comprise the same type of excipients in the tablet core and have the same tablet weight increase during coating of 5.0%.

Example 1 provides a dissolution profile similar to the commercial tofacitinib tablet XELJANZ XR® while the dissolution profile of example 2 is extremely slow and the dissolution profile of example 3 is too fast (no lag time).

1. A controlled release pharmaceutical tablet comprising:
a) A core comprising tofacitinib or a pharmaceutically acceptable salt thereof and a pH independent gelling control release polymer;
b) A coating in an amount of 1.5% to 10.0% w/w in relation to the total tablet weight comprising a water-insoluble polymer and a pore former in a weight ratio of from 90:10 w/w to 60:40 w/w respectively.

2. The tablet according to claim 1 such that tofacitinib is released from the tablet, in a controlled fashion so that at least 60% of tofacitinib is released after 4 hours and at least 80% of tofacitinib is released after 6 hours.

3. The tablet according to claim 1 wherein tofacitinib is present in an amount of from 3% to 15% by weight based on the total tablet core weight.

4. The tablet according to claim 1 wherein said pH independent gelling control release polymer is present in the core in an amount from 10% to 50% by weight to the total tablet core weight.

5. The tablet according to claim 1 wherein said pH independent gelling control release polymer has a viscosity of 10 cP or more when measured by rotational viscometer in a solution containing 2% of the polymer in distilled water at 22.5±0.5° C.

6. The tablet according to claim 1 wherein said pH independent gelling control release polymer in said core is selected from the group consisting of polyethylene oxide, hydroxypropyl methylcellulose, hydroxypropyl cellulose, polyvinyl alcohol, and combinations thereof.

7. The tablet according to claim 6, wherein said pH independent gelling control release polymer in said core is hydroxypropyl methylcellulose.

8. The tablet according to claim 1 wherein said water-insoluble polymer in said coating is selected from the group consisting of ethylcellulose, cellulose acetate, methacrylic ester copolymers, polyvinyl acetates, and combinations thereof.

9. The tablet according to claim 8, wherein said water-insoluble polymer in said coating is ethylcellulose.

10. The tablet according to claim 1 wherein said pore former in said coating is selected from the group consisting of hydroxypropyl cellulose, hydroxypropyl methylcellulose, methylcellulose, polyethylene glycol, polyethylene oxide, polyvinyl alcohol, povidone, maltodextrin, saccharides and combinations thereof.

11. The tablet according to claim 10, wherein said pore former in said coating is hydroxypropyl methylcellulose.

12. The tablet according to claim 1 wherein said core further comprises one or more excipients selected from the group consisting of diluent, glidant, lubricant, binder and buffering agent.

13. The tablet according to claim 1;
wherein said core comprises based on total weight of the core weight:
a) Tofacitinib or a pharmaceutically acceptable salt in an amount of from 3% to 15% w/w by weight;
b) Hydroxypropyl methylcellulose in an amount of from 10% to 50% w/w by weight;
c) Diluents in an amount of from 40% to 90% w/w by weight;
d) Glidant in an amount of from 0.2% to 1.0% w/w by weight;
e) Lubricant of from 0.05% to 5% w/w by weight;
and wherein said coating comprises ethylcellulose and hydroxypropyl methylcellulose in a weight ratio of from 90:10 w/w to 60:40 w/w.

14. The tablet according to claim 13 wherein said diluent is selected from the group comprising microcrystalline cellulose, lactose, phosphates, hydroxypropyl cellulose, starch and combinations thereof.

15. The tablet according to claim 1 wherein the tablet is coated such that the coating provides a tablet with 1.5% to 10% increase weight based on the total weight of the tablet core weigh.

16. The tablet according to claim 1 wherein tofacitinib is in the form of tofacitinib citrate.
